(19) United States
(12) Patent Application Publication
(10) Pub. No.: US 2013/0174964 A1
(43) Pub. Date: Jul. 11, 2013

(54) MICROFLUIDIC DEVICE AND METHOD OF FABRICATING THE SAME

(71) Applicant: SAMSUNG ELECTRONICS CO., LTD., Gyeonggi-do (KR)

(72) Inventors: Chin-sung PARK, Yongin-si (KR); Kak NAMKOONG, Seoul (KR); Young-sun LEE, Seongnam-si (KR)

(73) Assignee: SAMSUNG ELECTRONICS CO., LTD., Gyeonggi-do (KR)

(21) Appl. No.: 13/782,562

(22) Filed: Mar. 1, 2013

Related U.S. Application Data
(62) Division of application No. 12/184,317, filed on Aug. 1, 2008.

(30) Foreign Application Priority Data
Jan. 2, 2008 (KR) 10-2008-0000165

(51) Int. Cl. C12M 3/06

(52) U.S. Cl. CPC C12M 23/16
USPC 156/73.1; 156/60

(57) ABSTRACT

Provided are a microfluidic device that performs a biochemical reaction using a small amount of a biochemical fluid and detects the result thereof, and a method of fabricating the same. The microfluidic device includes: a substrate which comprises a chamber that is formed as a concave groove and accommodates a fluid in the bottom surface of the substrate, and is formed of polymer; and a film welded on the bottom surface of the substrate to seal the chamber so that the chamber is not open at the bottom surface of the substrate, and formed of polymer. The method of fabricating a microfluidic device includes: preparing a substrate which comprises a chamber that is formed as a concave groove and accommodates a fluid in the bottom surface of the substrate, and is formed of polymer; and welding a film on a bottom surface of the substrate to seal the chamber so that the chamber is not opened at the bottom surface of the substrate, the film being formed of polymer.

### CROSS-REFERENCE TO RELATED PATENT APPLICATIONS

This is a Divisional of application Ser. No. 12/184,317 filed on Aug. 1, 2008, which application claims the benefit of Korean Patent Application No. 10-2008-0000165, filed on Jan. 2, 2008, in the Korean Intellectual Property Office, the disclosure of which is incorporated herein in its entirety by reference.

### BACKGROUND OF THE INVENTION

1. Field of the Invention

The present invention relates to microfluidics, and more particularly, to a microfluidic device which performs a biochemical reaction using a small amount of a biochemical fluid and detects the result thereof, and a method of fabricating the same.

2. Description of the Related Art

In microfluidic engineering, research is being actively conducted on microfluidic devices having various functions such as performing biochemical reactions using biochemical fluids such as blood and urine and detecting the results of the reactions. Such microfluidic devices include a chip-formed device known as a lab-on-a-chip, or a disk-shaped device that is rotatable and known as a lab-on-a-disk. A microfluidic device includes a chamber in which a fluid is accommodated and a channel that is connected to the chamber.

FIG. 1 is a cross-sectional view of a conventional microfluidic device 10 for a polymerase chain reaction (PCR).

Referring to FIG. 1, the conventional microfluidic device 10 includes a lower substrate 11 and an upper substrate 15 that are attached to each other and a chamber 20 inside. The microfluidic device 10 is used to perform a polymerase chain reaction (PCR) using a biochemical fluid accommodated in the chamber 20. In this regard, the microfluidic device 10 and can also be referred to a “PCR chip.” In order to perform PCR, the biochemical fluid accommodated in the microfluidic device 10 needs to be heated in regular cycles, and thus the process of PCR is also known as “thermal cycling”. A PCR using the microfluidic device 10 can be completed in a shorter time than a conventional PCR process in which a biochemical fluid is injected into a tube to perform PCR. Thus the frequency of use of microfluidic devices such as the microfluidic device 10 is increasing.

The lower substrate 11 is formed of silicon (Si) having excellent thermal conductivity so that thermal conduction can occur in regular cycles and at high speed. The result of a PCR occurring in the biochemical fluid accommodated in the chamber 20 is detected using a fluorescence detection method, and thus the upper substrate 15 of the microfluidic device 10 is formed of a transparent glass. The fluorescence detection method can be used to detect the process of the biochemical reaction in real-time by detecting a fluorescence signal emitting light in a biochemical fluid. However, as described above, since the lower substrate 11 is formed of Si and the upper substrate 15 is formed of glass, the manufacturing cost of the microfluidic device 10 is increased.

### SUMMARY OF THE INVENTION

The present invention provides a microfluidic device that is formed of a polymer material to reduce manufacturing cost, and in which fast and efficient thermal conduction can be performed, and a method of fabricating the same.

According to an aspect of the present invention, there is provided a microfluidic device comprising: a substrate which comprises a chamber that is formed as a concave groove and accommodates a fluid in the bottom surface of the substrate, and is formed of polymer; and a film welded on the bottom surface of the substrate to seal the chamber so that the chamber is not open at the bottom surface of the substrate, and formed of polymer.

The welding may be ultrasonic welding or thermal welding.

The substrate may further comprise a welding peak on the bottom surface of the substrate such that the welding peak is welded on the film by ultrasonic vibration.

The height of the welding peak may be smaller than or equal to the thickness of the film.

The thickness of the film may be 30-100 μm.

The substrate and the film may be formed of the same material.

Polymer, as a material of the substrate, may be one material selected from the group consisting of polypropylene (PP), polycarbonate (PC), polyethylene (PE), polyethylene terephthalate (PET), polymethylmethacrylate (acrylic)(PMMA) or cyclic olefin copolymer (COC).

Polymer, as a material of the film, may be one material selected from the group consisting of polypropylene (PP), polycarbonate (PC), polyethylene (PE), polyethylene terephthalate (PET), polymethylmethacrylate (acrylic)(PMMA) or cyclic olefin copolymer (COC).

The substrate may further comprise a channel formed in the bottom surface of the substrate to connect with the chamber.

The substrate may further comprise an inlet hole in order to inject a fluid or an outlet hole in order to discharge the air inside the chamber when the fluid is injected into the inlet hole, each of the two holes being connected to the channel and open to a top surface of the substrate.

The substrate may further comprise at least one handling portion for a user to handle the microfluidic device.

The microfluidic device may be used to perform a biochemical reaction involving a biochemical fluid, and the substrate may further comprise at least one aligning portion as a criterion for alignment when the microfluidic device is aligned with and installed on a device for performing the biochemical reaction.

The microfluidic device may be used in a PCR (polymerase chain reaction) of a biochemical fluid.

The substrate may be transparent so that the PCR can be detected in real-time by using an optical method.

The transmittance of the substrate with respect to incident light in a band of visible rays may be 90%-100%.

According to another aspect of the present invention, there is provided a method of fabricating a microfluidic device, the method comprising: preparing a substrate which comprises a chamber that is formed as a concave groove and accommodates a fluid in the bottom surface of the substrate, and is formed of polymer; and welding a film on a bottom surface of the substrate to seal the chamber so that the chamber is not opened at the bottom surface of the substrate, the film being formed of polymer.

The welding of the film may comprise adhering the film onto the bottom surface of the substrate either by ultrasonic welding or thermal welding.

The preparing of the substrate may further comprise forming at least one welding peak to be welded on the film on the bottom surface of the substrate, and the welding of the film may comprise contacting the welding peak with the film, and welding the welding peak on the film by ultrasonic vibration.

### BRIEF DESCRIPTION OF THE DRAWINGS

The above and other features and advantages of the present invention will become more apparent by describing in detail exemplary embodiments thereof with reference to the attached drawings in which:

FIG. 1 is a cross-sectional view showing an example of a conventional microfluidic device for performing a polymerase chain reaction (PCR);

FIG. 2 is a perspective view of a microfluidic device according to an embodiment of the present invention;

FIG. 3 is an exploded perspective view of the microfluidic device of FIG. 2; and

FIGS. 4A and 4B are cross-sectional views taken along line IV-IV of FIGS. 2 and 3, respectively, and sequentially illustrate a method of fabricating the microfluidic device of FIG. 2.

### DETAILED DESCRIPTION OF THE INVENTION

The present invention will now be described more fully with reference to the accompanying drawings, in which exemplary embodiments of the invention are shown.

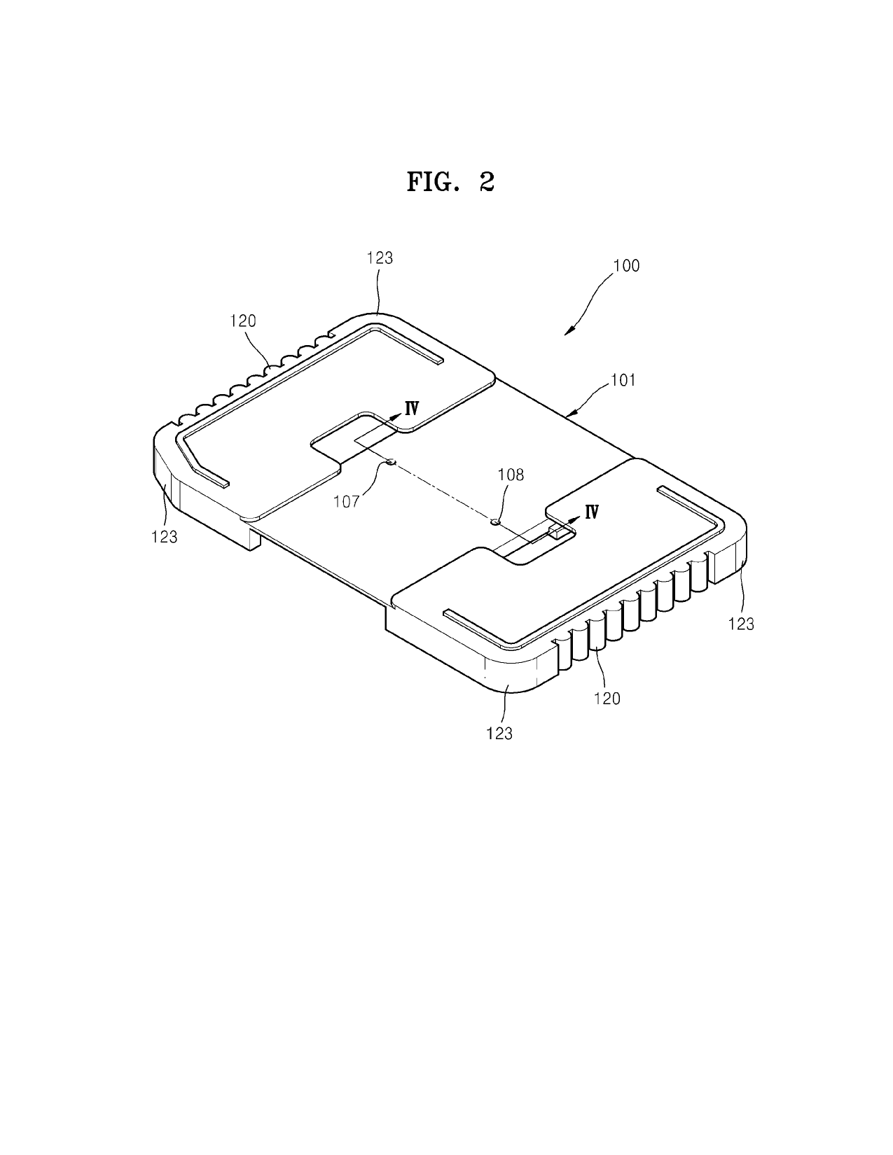

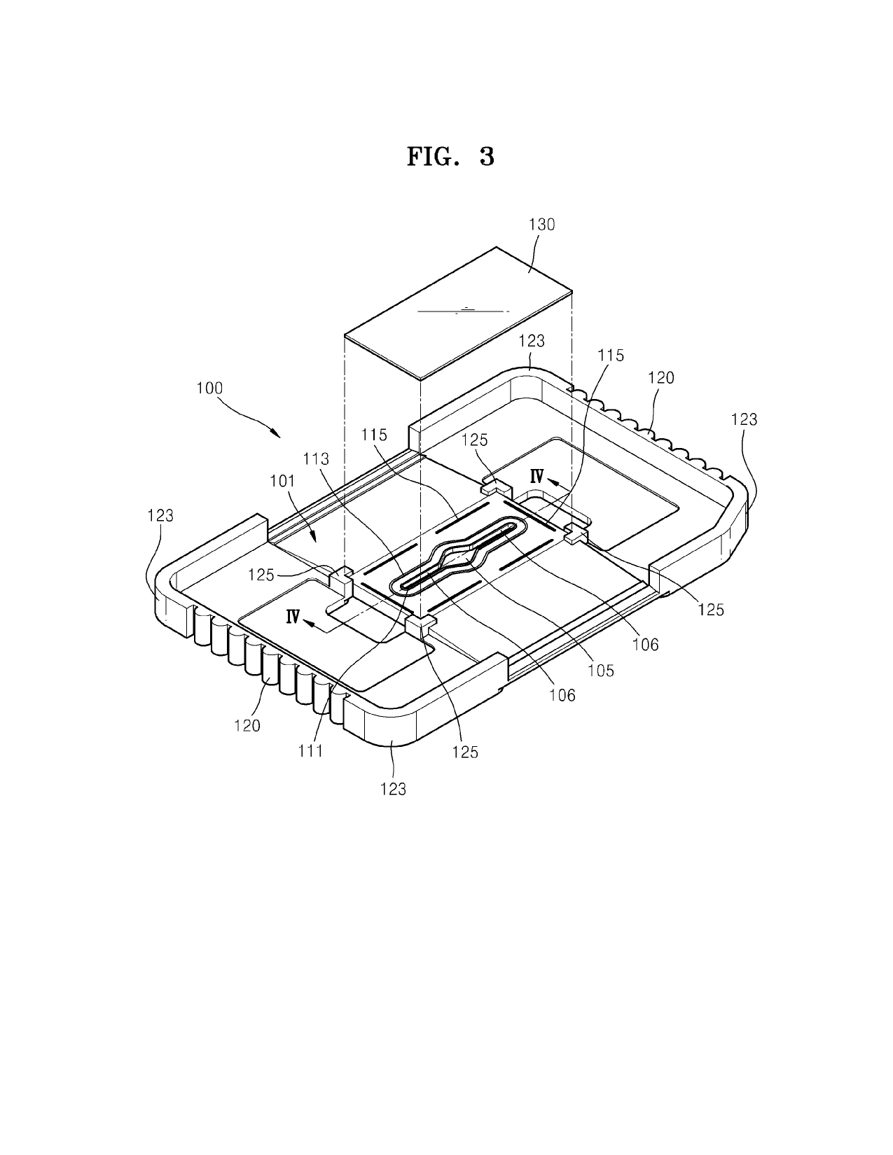

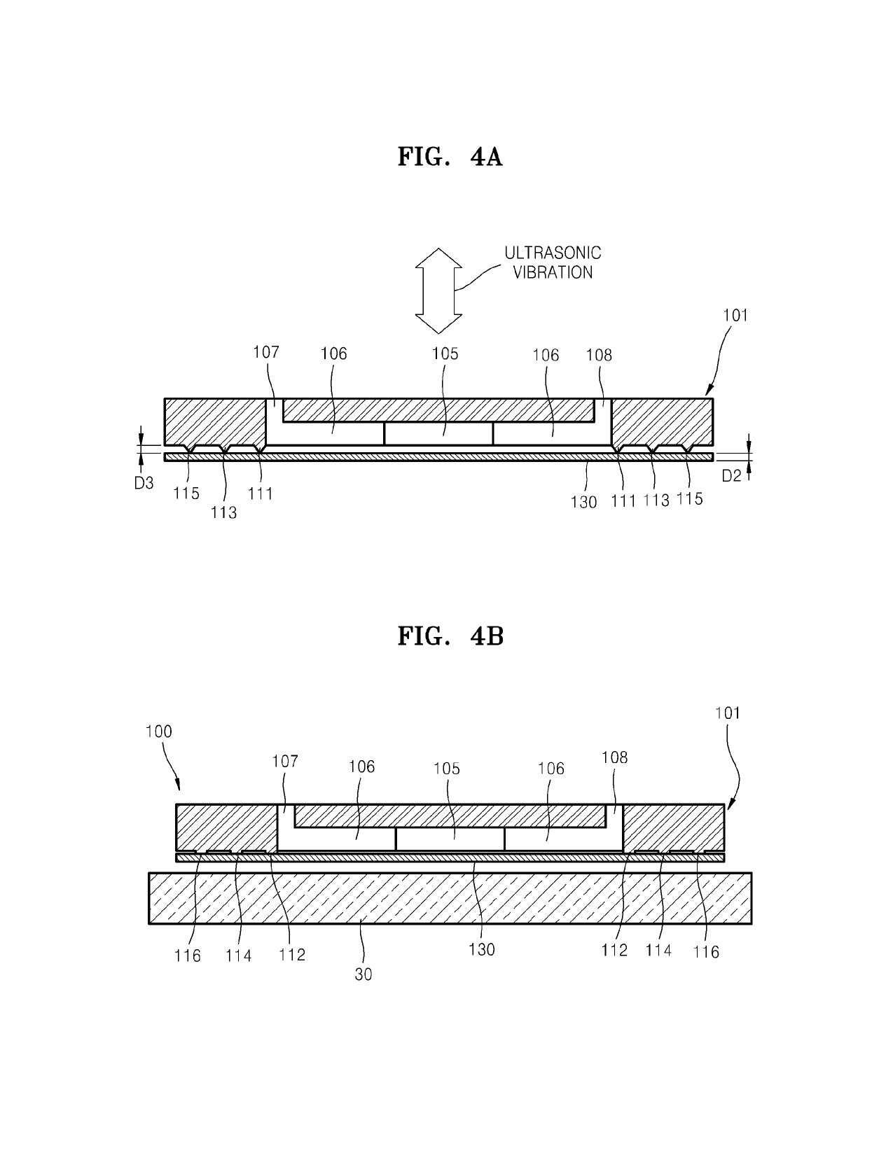

FIG. 2 is a perspective view of a microfluidic device 100 according to an embodiment of the present invention, FIG. 3 is an exploded perspective view of the microfluidic device 100 of FIG. 2, and FIGS. 4A and 4B are cross-sectional views taken along line IV-IV of FIGS. 2 and 3, respectively, and sequentially illustrate a method of fabricating the microfluidic device 100 of FIG. 2.

Referring to FIGS. 2 and 3, the microfluidic device 100 according to the present embodiment of the present invention comprises a substrate 101 and a film 130 that is welded on a bottom surface of the substrate 101. The microfluidic device 100 is designed to perform a polymerase chain reaction (PCR) however the present embodiment of the present invention is not limited to such usage. The substrate 101 is formed of polymer, which is cheaper and is more easily processed than silicon (Si) or glass. Such polymer may be one that does not react with a biochemical fluid accommodated in the microfluidic device 100, i.e., polymer having a chemical resistant property. In addition, a polymer having a stable surface on which materials included in the biochemical fluid are not absorbed may be used. In the present embodiment, the polymer used as a material to form the substrate 101 is polypropylene (PP), polycarbonate (PC), polyethylene (PE), polyethylene terephthalate (PET), polymethylmethacrylate (acrylic) (PMMA) or cyclic olefin copolymer (COC).

The substrate 101 comprises a chamber 105 and a channel 106 which are connected to each other and respectively formed as a concave groove in a bottom surface of the substrate 101. The chamber 105 and the channel 106 may be formed when the substrate 101 is formed by injection molding a liquid resin. The chamber 105 is a place in which the PCR involving the biochemical fluid is induced and the result of the PCR is optically, more specifically, fluorescence detected.

The substrate 101 comprises an inlet hole 107 and an outlet hole 108, which are connected to the channel 106 and are opened to a top surface of the substrate 101. The inlet hole 107 is used to inject the biochemical fluid into the microfluidic device 100, and the outlet hole 108 is used to discharge the air inside the chamber 105 when the biochemical fluid is injected into the microfluidic device 100. The inlet hole 107 and the outlet hole 108 may be formed by mechanically processing the substrate 101.

The substrate 101 may be transparent so as to detect a biochemical reaction such as the PCR in real-time through an optical method. On the other hand, a method of optically detecting the biochemical reaction such as the PCR involves radiating visible rays on the biochemical fluid accommodated in the chamber 105 and detecting and analyzing fluorescence or phosphorescence of the biochemical fluid due to the radiation of the visible rays. However, when self-light emission of the substrate 101 with respect to incident light in a band of visible rays is large, errors may be easily generated during optical detection of the biochemical reaction, and the reliability of the result of the optical detection is lowered. Thus, the substrate 101 may have low self-light emission with respect to incident light in the band of visible rays and a large transmittance of 90%-100%.

The substrate 101 may further comprise, apart from the chamber 105, the channel 106, the inlet hole 107, and the outlet hole 108, which are directly used to perform the biochemical reaction, configurations at its peripheral for user convenience. Specifically, the substrate 101 may comprise handling portions 120 for the user to handle when the microfluidic device 100 is to be transported or aligned or when handling the microfluidic device 100 to inject a fluid into the inlet hole 107. Furthermore, the substrate 101 may comprise aligning portions 123 disposed at least one corner of the microfluidic device 100, and as the criterion for alignment when the microfluidic device 100 is aligned with and installed on a device which is provided in order to perform the biochemical reaction or optically detect the result of the biochemical reaction. The handling portions 120 and the aligning portions 123 are formed when the substrate 101 is formed by injection molding a liquid resin, like the procedure for forming the chamber 105 and the channel 106.

The film 130 is sealed onto the bottom of the substrate 101 so that the chamber 105, the channel 106, the inlet hole 107, and the outlet hole 108 are not opened at the bottom surface of the substrate 101. The film 130 is welded on the bottom surface of the substrate 101. As such, a biochemical fluid (not shown) injected into the microfluidic device 100 through the inlet hole 107 may not flow through the channel 106 and the chamber 105 but may be accommodated in the channel 106 and the chamber 105. The film 130 may be welded on the bottom of the substrate 101 either by ultrasonic welding or thermal welding.

The film 130 may be formed of polymer. Such polymer may be one that does not react with a biochemical fluid accommodated in the microfluidic device 100, i.e., polymer having a chemical resistant property. In addition, a polymer having a stable surface on which materials included in the biochemical fluid are not absorbed may be used. For example, the polymer used as a material to form the film 130 is polypropylene (PP), polycarbonate (PC), polyethylene (PE), polyethylene terephthalate (PET), polymethylmethacrylate (acrylic) (PMMA) or cyclic olefin copolymer (COC). The film 130 and the substrate 101 may also be formed of the same material. For example, both of the substrate 101 and the film 130 may be formed of transparent COC.

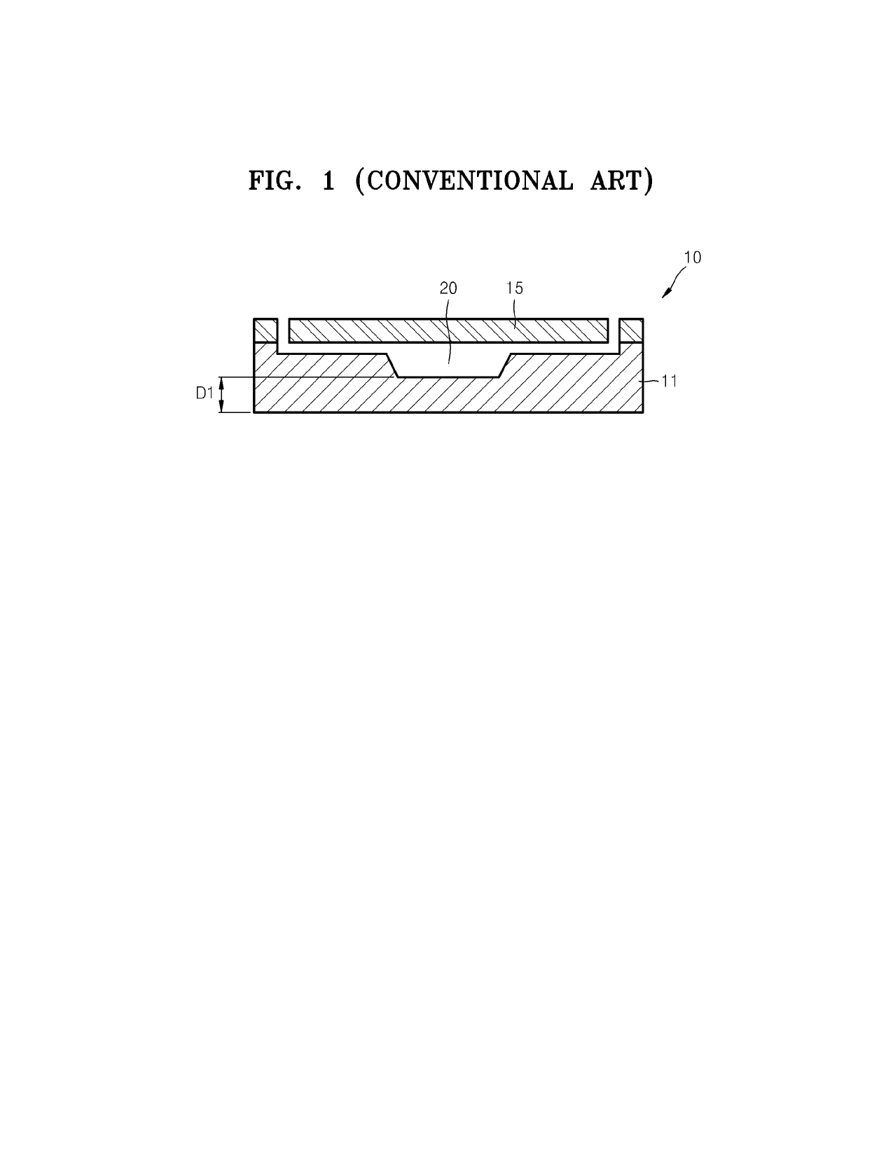

As described in detail with reference to FIG. 1, the procedure for performing the PCR is also called a “thermal cycling procedure”, and in the conventional art, a lower plate 11 (see FIG. 1) that contacts with a microheater 30 (see FIG. 4B) is formed of silicon (Si) so that thermal conduction can be performed according to a fast and exact period. Silicon (Si) has a thermal conductivity k of 157 W/m/K, which is much higher than polymer. Thus, when the thickness D2 (see FIG. 4A) of the film 130 of the microfluidic device 100 according to the present invention is set to be the same as the thickness D1 (see FIG. 1) from the bottom surface of the silicon lower plate 11 to the bottom of the chamber 20 of the conventional microfluidic device 10, a microfluidic device used for a PCR cannot be fabricated. Consequently, the thickness D2 of the film 130 is much smaller than the thickness D1 and thus is proper.

A transient thermal conduction equation is non-dimensionalized as the following equation 1. A non-dimensionalization coefficient of equation 1 is defined by equation 2.

$$\begin{matrix}\frac{\partial {θ}^{*}}{\partial {F}_{o}} = \frac{{\partial }^{2} {θ}^{*}}{\partial {x}^{* 2}} & ( 1 ) \\ {θ}^{*} = \frac{T - {T}_{∞}}{{T}_{i} - {T}_{∞}} , \\ {x}^{*} = \frac{x}{L} , \\ {F}_{o} = \frac{t}{{L}^{2} / α} , & ( 2 )\end{matrix}$$

where L²/α is a thermal conduction time scale. L is the thickness of a thermal conductor that contacts a heater, and α is thermal duffusivity. The thermal conduction time scale is defined using equation 3.

conduction time scale=ρCₚL²/k (3)

where k is thermal conductivity of a thermal conductor that contacts a heater, and ρ is density of the thermal conductor, and Cₚ is specific heat of the thermal conductor.

When the thickness D2 of the film 130 is set so that a difference in the thermal conduction time scale between the lower plate 11 of the conventional microfluidic device 10 (see FIG. 1) formed of silicon (Si) and the film 130 of the microfluidic device 100 according to the present invention is not large, in spite of a large difference in thermal conductivity k therebetween, the microfluidic device 100 that can be used for the biochemical reaction such as the PCR can be fabricated.

The inventor calculated a thermal conduction time scale according to the thickness D2 of the film 130 formed of COC by using equation 3. A thermal conductivity k of the film 130 is 0.135 W/m/K, its density ρ is 1020 kg/m³, and its specific heat Cₚ is 1000 J/kg/K. Therefore, when the thickness D2 of the film 130 was changed from 10 μm to 100 μm, the thermal conduction time scale changed from 0.756 msec to 75.6 msec.

On the other hand, the thickness D1 from the bottom surface of the chamber 20 to the bottom surface of the lower plate 11, of the conventional microfluidic device 10 of FIG. 1 is 350 μm, a thermal conductivity k of the lower plate 11 is 157 W/m/K, its density ρ is 2329 kg/m³, its specific heat Cₚ is 700 J/kg/K, and its thermal conduction time scale is 1.27 msec.

When the thickness D2 of the film 130 of the microfluidic device 100 according to the present invention is approximately 10 μm, a thermal conduction time scale of the film 130 is better than the conventional microfluidic device 10 (see FIG. 1). However, the physical strength of the film 130 is too low and the film 130 cannot withstand high temperature and high pressure conditions during a biochemical reaction and thus very careful handling is needed. When the thickness D2 of the film 130 is equal to or greater than 30 μm, the film 130 has a physical strength in which the film 130 can stand such conditions during a biochemical reaction such as the PCR or the like. On the other hand, when the thickness D2 of the film 130 is greater than 100 μm, its thermal conduction time scale is too large and the PCR cannot be completed within an equal time as the conventional microfluidic device 10 (see FIG. 1). Thus, the thickness of the film 130 may be 30-100 μm.

Hereinafter, the method of fabricating the microfluidic device 100 by using ultrasonic welding will be described with reference to FIGS. 4A and 4B.

Referring to FIG. 4A, the substrate 101 and the film 130 are prepared. The substrate 101 may be formed by injection molding using a polymer resin, and first, second, and third welding peaks 111, 113, and 115 are formed in the vicinity of the chamber 105 and the channel 106 formed in the bottom surface of the substrate 101. As illustrated in FIG. 3, the first welding peak 111 is formed to correspondingly encompass the chamber 105 and the channel 106 without a portion separated so as to be most adjacent to the chamber 105 and the channel 106. The second welding peak 113 is formed to encompass the first welding peak 111 without a portion separated at the outside of the first welding peak 111. The third welding peak 115 is formed to face the outer circumference part of the film 130, at the outside of the second welding peak 113.

Next, the substrate 101 and the film 130 are close to each other so that the tips of the first, second, and third welding peaks 111, 113, and 115 contact the film 130. The film 130 is aligned on a film alignment protrusion 125 of the substrate 101. Next, the substrate 101 is vibrated with ultrasonic frequencies. As such, the tips of the first, second, and third welding peaks 111, 113, and 115 start to meld and weld on the film 130. All of the first, second, and third welding peaks 111, 113, and 115 are melted and their tips are removed, and thus, first, second, and third welding traces 112, 114, and 116 are left, as illustrated in FIG. 4B. As such, the first substrate 101 and the film 130 are sealed and adhered to each other so that a fluid may not leak out. In the present embodiment, the height D3 of each of the first, second, and third welding peaks 111, 113, and 115 is equal to or less than the thickness D2 of the film 130. The method of fabricating the microfluidic device 100 using ultrasonic welding has been described with reference to FIGS. 4A and 4B, however, the present invention is not limited to this, and thus, another microfluidic device according to the present invention may also be fabricated using welding by heating and pressurization.

A PCR that is performed in the chamber 105 of the microfluidic device 100 according to the present invention can be analyzed in real-time by detecting fluorescence signals that are emitted from the biochemical fluid accommodated in the chamber 105. In this way, a method of analyzing a biochemical reaction by detecting fluorescence signals is called fluorescence detection. Various fluorescence detection methods used in PCR analysis have been developed, and such methods include a method of using a dye, such as SYBR Green I, that emits fluorescence when the dye is bound to double stranded deoxyribonucleic acid (DNA) generated by a PCR and a method of using a phenomenon that fluorescence is generated as the bond between a fluorophore and a quencher at the end of a probe is broken, and so forth. Fluorescence detection of a PCR is well known to one of ordinary skill in the art and a detailed description thereof will be omitted. The inventors have obtained a similar analysis result on both the conventional microfluidic device 10 (see FIG. 1) and the microfluidic device 100 formed of COC according to the present invention. Thus, the inventors have verified that the microfluidic device 100 according to the present invention can be applied for PCR analysis.

While the present invention has been particularly shown and described with reference to exemplary embodiments thereof, it will be understood by one of ordinary skill in the art that various changes in form and details may be made therein without departing from the spirit and scope of the present invention as defined by the following claims.

What is claimed is:

1. A method of fabricating a microfluidic device, the method comprising:
preparing a substrate which comprises a chamber that is formed as a concave groove and accommodates a fluid in the bottom surface of the substrate, and is formed of polymer; and
welding a film on a bottom surface of the substrate to seal the chamber so that the chamber is not opened at the bottom surface of the substrate, the film being formed of polymer.

2. The method of claim 16, wherein the welding of the film comprises adhering the film onto the bottom surface of the substrate either by ultrasonic welding or thermal welding.

3. The method of claim 16, wherein the preparing of the substrate further comprises forming at least one welding peak to be welded on the film on the bottom surface of the substrate, and the welding of the film comprises contacting the welding peak with the film, and welding the welding peak on the film by ultrasonic vibration.

4. The method of claim 18, wherein the height of the welding peak is smaller than or equal to the thickness of the film.

5. The method of claim 16, wherein the thickness of the film is 30-100 μm.

6. The method of claim 16, wherein the substrate and the film are formed of the same material.

7. The method of claim 16, wherein polymer, as a material of the substrate, is one material selected from the group consisting of polypropylene, polycarbonate, polyethylene, polyethylene terephthalate, PMMA polymethylmethacrylate or cyclic olefin copolymer.

8. The method of claim 16, wherein polymer, as a material of the film, is one material selected from the group consisting of polypropylene, polycarbonate, polyethylene, polyethylene terephthalate, polymethylmethacrylate or cyclic olefin copolymer.
